(19) United States
(12) Patent Application Publication
(10) Pub. No.: US 2024/0139054 A1
(43) Pub. Date: May 2, 2024

(54) Adjustable Exoskeleton Foot-Supporting Appendage for Variable Human Kinematic Compatibility

(71) Applicant: Sarcos Corp., Salt Lake City, UT (US)

(72) Inventors: Fraser M. Smith, Salt Lake City, UT (US); Marc X. Olivier, Salt Lake City, UT (US); Michael Myers, Holladay, UT (US)

(21) Appl. No.: 17/979,689

(22) Filed: Nov. 2, 2022

(51) Int. Cl. A61H 3/00

(52) U.S. Cl. CPC A61H 3/00; A61H 2201/1215; A61H 2201/165; A61H 2201/5061

(57) ABSTRACT

A foot-supporting appendage of a wearable robotic exoskeleton configured to support a foot of a user wearing the robotic exoskeleton. The foot-supporting appendage can include a rearward support configured to support a hind foot portion of a foot of the user. The foot-supporting appendage can further include a frontward support configured to connect to the rearward support and configured to support a forefoot portion of the foot of the user. A size of the foot-supporting appendage is adjustable to accommodate a respective foot of a user of a plurality of users having different sizes of feet.

### BACKGROUND

In robots configured to be used as wearable robotic exoskeletons, a biological organism such as a human user dons and controls the exoskeleton for a variety of purposes and to perform various tasks in a variety of different environments and applications. In the wide array of environments and applications in which wearable robotic exoskeletons are used, many different users of many different sizes and shapes may need to use a wearable exoskeleton. Wearable robotic exoskeletons are complex and expensive to design, build, and use. As such, it is cost prohibitive for manufacturers to design and produce wearable exoskeletons of every shape and size to fit every possible user. It can also be cost prohibitive and inefficient for consumers to purchase wearable exoskeletons that may be custom sized to fit only a single specific person. To avoid the problems described above, improved designs of wearable robotic skeletons to accommodate different users continue to be developed.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1 illustrates an isometric view of a robot in the form of a wearable robotic exoskeleton in accordance with an example of the present disclosure.

FIGS. 2A, 2B, 2C, and 2D respectively illustrate an isometric view, a top view, a left side view, and a right side view of a foot-supporting appendage operable with the wearable robotic exoskeleton of FIG. 1 in accordance with an example of the present disclosure, where the foot-supporting appendage includes a first frontward support.

FIG. 3 illustrates the first frontward support of the exemplary foot-supporting appendage of FIGS. 2A-2D being interchangeable with the second frontward support of the exemplary foot-supporting appendage of FIGS. 4A-4D.

FIGS. 4A, 4B, 4C, and 4D respectively illustrate an isometric view, a top view, a left side view, and a right side view of a foot-supporting appendage operable with the wearable robotic exoskeleton of FIG. 1 in accordance with an example of the present disclosure, where the foot-supporting appendage includes a second frontward support different from the first frontward support.

FIGS. 5A, 5B, 5C, and 5D respectively illustrate an isometric view, a top view, a left side view, and a right side view of a foot-supporting appendage operable with the wearable robotic exoskeleton of FIG. 1 in accordance with an example of the present disclosure where the foot-supporting appendage includes a third frontward support different from the first and second frontward supports.

FIG. 6 illustrates a top view comparison of the foot-supporting appendage of the robotic exoskeleton of FIG. 1, and each of the different sized and configured first, second and third frontward supports of FIGS. 2A-2D, 4A-4D, and 5A-5D, respectively.

FIG. 7 illustrates a side view comparison of the foot-supporting appendage of the robotic exoskeleton of FIG. 1, and each of the different sized and configured first, second and third frontward supports of FIGS. 2A-2D, 4A-4D, and 5A-5D, respectively.

FIG. 8A illustrates an isometric view of a foot-supporting appendage operable with the robotic exoskeleton of FIG. 1 in accordance with an example of the present disclosure.

FIG. 8B illustrates a side view of the foot-supporting appendage of FIG. 8A.

FIG. 8C illustrates a side view of a foot-supporting appendage operable with the robotic exoskeleton of FIG. 1 in accordance with an example of the present disclosure.

FIG. 8D illustrates a side view of a foot-supporting appendage operable with the robotic exoskeleton of FIG. 1 in accordance with an example of the present disclosure.

FIG. 9 illustrates a top view of a foot-supporting appendage of a wearable exoskeleton in accordance with an example of the present disclosure.

FIG. 10A illustrates a side view of a foot-supporting appendage of a wearable exoskeleton in a retracted configuration in accordance with an example of the present disclosure.

FIG. 10B illustrates a side view of a foot-supporting appendage of a wearable exoskeleton in an extended configuration in accordance with an example of the present disclosure.

FIG. 11 illustrates a side view of a foot-supporting appendage of a wearable exoskeleton in accordance with an example of the present disclosure.

FIG. 12 illustrates a method of adjusting a size of a foot-supporting appendage in accordance with an example of the present disclosure.

FIG. 13 illustrates a method of adjusting a size of a foot-supporting appendage in accordance with an example of the present disclosure.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness can in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” can be either abutting or connected. Such elements can also be near or close to each other without necessarily contacting each other. The exact degree of proximity can in some cases depend on the specific context.

An initial overview of the inventive concepts are provided below and then specific examples are described in further detail later. This initial summary is intended to aid readers in understanding the examples more quickly, but is not intended to identify key features or essential features of the examples, nor is it intended to limit the scope of the claimed subject matter.

Disclosed herein is a foot-supporting appendage of a wearable robotic exoskeleton configured to support a foot of a user wearing the robotic exoskeleton. The foot-supporting appendage can comprise a rearward support having a support surface, and configured to support a hind foot portion of a foot of the user. The foot-supporting appendage can further comprise a frontward support comprising a support surface, and configured to couple to the rearward support and to support a forefoot portion of the foot of the user. A size of the foot-supporting appendage is adjustable to accommodate a foot of a user of a plurality of users having different sizes of feet.

Further disclosed herein is a wearable robotic exoskeleton system. The wearable robotic exoskeleton system can include a foot-supporting appendage configured to support a foot of a user wearing the robotic exoskeleton. The foot-supporting appendage can include a rearward support configured to support a hind foot portion of a foot of the user. The foot-supporting appendage can further include a frontward support configured to connect to the rearward support and configured to support a forefoot portion of the foot of the user. A size of the foot-supporting appendage is adjustable to accommodate a foot of a user of a plurality of users having different sizes of feet.

Further disclosed herein is an adjustable foot-supporting appendage system for a wearable robotic exoskeleton. The adjustable foot-supporting appendage system can include a foot-supporting appendage of a wearable robotic exoskeleton configured to support a foot of a user wearing the robotic exoskeleton. The foot-supporting appendage can include a rearward support configured to support a hind foot portion of a foot of the user. The foot-supporting appendage can further include a plurality of frontward supports configured to interchangeably connect to the rearward support and configured to support a forefoot portion of the foot of the user. Each of the plurality of frontward supports are associated with a foot size of a user. A size of the foot-supporting appendage is adjustable by removably coupling one of the plurality of frontward supports to the rearward support, the one of the plurality of frontward supports being a size corresponding to a size of foot of the user.

Further disclosed herein is a method for modifying or adjusting a size of a foot-supporting appendage of a wearable robotic exoskeleton. The appendage can include a rearward support configured to support a hind foot portion of a foot of a user, and a frontward support configured to connect to the rearward support and configured to support a forefoot portion of the foot of the user. The method can include identifying a rearward support configured to support a hind foot portion of a foot of a user, identifying a frontward support configured to couple to the rearward support, and to support a forefoot portion of the foot of the user, and sizing the foot-supporting appendage to a size of the foot of the user.

To further describe the present technology, examples are now provided with reference to the figures.

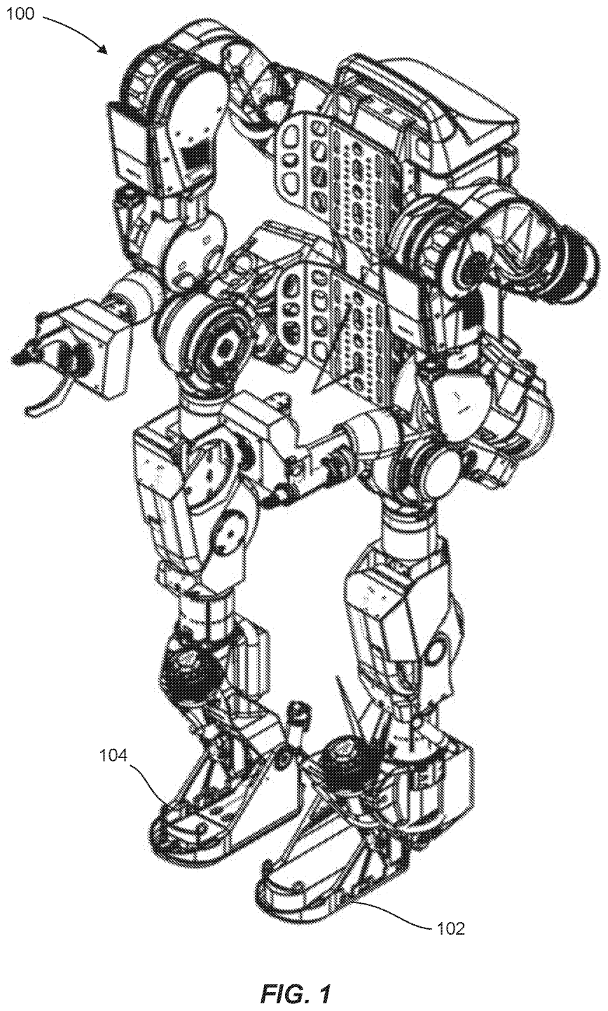

FIG. 1 illustrates an exemplary robot or robotic system in the form of a wearable robotic exoskeleton 100 that is gait-capable. The term “wearable” is intended to refer to any robot of any configuration that is configured to receive one or more parts of a user's body. A robot can be considered wearable when any part or parts of the user's body is/are in contact with or interfaces with any part of the robot to control or move the robot. The robot can further be considered wearable when a part of a user's body interfaces with and/or contacts the robot in a resting or passive configuration. Robots that are wearable can comprise a robot that receives a part of the user's body, no matter how small or large, such as an arm, hand, finger, leg, foot, toe, head, neck, torso, pelvis, or any portion or combination of said parts.

The term “gait-capable” is intended to refer to one or more types of movements relative to ground (or a surface modeling ground) that a robot can perform during operation. These can include, but are not limited to, gait-based locomotion movements, capabilities, or operations, as well as gait-associated and/or stance-associated movements, capabilities or operations that a robot with any number of jointed appendages (i.e., robotic limbs or legs having a ground contacting portion, such as feet or other ground contacting structures/assemblies) can perform during operation. Example gait-based locomotion or gait-associated movements, capabilities or operations can include, but are not limited to a walking gait, running gait, jumping, hopping, and others as will be apparent to those skilled in the art. Example stance-associated movements or capabilities or operations can include, but are not limited to standing (i.e., where the robot is capable of operating in an erect position), squatting, toe stance (i.e., support of the robot with only the forefoot section of a foot), transitioning movements between possible stances for the robot and others as will be apparent to those skilled in the art.

The robotic exoskeleton 100 shown is one example of such a robot, the robotic exoskeleton 100 being a biped robot capable of bipedal locomotion, as well as one or more gait-associated and/or stance-associated operations, such as running, walking, jumping, hopping, standing, squatting, balancing on one leg, and others. Robots that are gait-capable and/or are capable of one or more gait-associated and/or stance associated operations can comprise bipedal robots, quadruped robots, and any others with any number of appendages (one or more appendages) in contact with the ground that are capable of locomotion and/or positioning of the robot. Although many of the examples described herein are described as gait-capable, it will be appreciated that the following disclosure equally applies to robots that are one or more of gait-capable, capable of gait-associated operations, or capable of stance-associated operations.

Being an exoskeleton type, the robotic exoskeleton 100 is designed and configured to be operated and to move in accordance with a bipedal gait cycle during locomotion that corresponds to human gait movements, namely a human gait cycle, as well as other capabilities that can be performed by a human (e.g., standing, squatting, jumping, hopping, sitting, running, pivoting, tilting, shifting weight on one or more appendages). Indeed, the robotic exoskeleton 100 can be configured to be operated to facilitate one or more gait patterns, as well as to facilitate one or more capabilities. For example, the robotic exoskeleton 100 can be configured to be operated to facilitate at least one of a walking gait pattern, a running gait pattern, as well as to facilitate operation of the robotic exoskeleton 100 in a standing or other stance position, to achieve a squatting function, to achieve a toe stance function, and others.

Although the discussion below will focus on the robotic exoskeleton 100 shown, this is not intended to be limiting in any way as the adjustable appendage technology discussed herein can be utilized on any gait-capable or non-gait capable robot or robotic device that is capable of receiving a human foot in an appendage of the robot. In one example, contemplated robots or robotic devices can be any robot or robotic device capable of one or more of gait movements (i.e., gait movements for locomotion) gait-associated operations, and/or stance-associated operations (e.g., standing, squatting, toe stance). In another example, contemplated robots or robotic devices can be any robot capable of only one of these. More specifically, and as further discussed above, a gait-capable robot can comprise a robot or robotic device capable of gait movements for locomotion where the robot moves by using contact between a ground or surface and an appendage to propel the robot in a given direction on and about the ground or surface. With respect to a walking or running gait pattern, to perform this type of gait, the appendage of the gait-capable robot can contact a surface, exert force on the surface in a motion that propels the robot in a desired direction, be removed from the surface, and swing in a direction of desired motion in preparation for another surface contact. This pattern can be repeated in a gait cycle. In this example, the gait-capable robot can be mobile and self-propelled, such as with power actuated joints, a propulsion system (e.g., appendages and motors) and other components and systems.

Such a robot can also be capable of other types of gait patterns (e.g., hopping, jumping gait patterns), as well as other gait-associated and/or stance-associated operations (e.g., standing, squatting, toe stance, pivoting, moving, shifting weight). In still another example, robots or robotic devices that are only capable of one or more or certain gait patterns are also contemplated. Although examples described below are described as gait-capable robots, it will be appreciated that the exemplary robots described herein may be gait-capable, stance capable, or capable of one or more gait-associated or stance-associated operations or any combination of the above. Additionally, the exemplary robots described herein that utilize a foot receiving appendage can be stationary and/or non-gait capable robots that receive a part of a user's body.

Although the discussion below will focus on the gait-capable, wearable, robotic exoskeleton 100 shown, this is not intended to be limiting in any way as the adjustable appendage technology discussed herein can be utilized on any robot or robotic device capable of receiving a body part of a user. In one example, contemplated robots or robotic devices can be any robot or robotic device that receives a foot or feet of a user.

Turning now to the example wearable robotic exoskeleton 100 shown, it can be seen that the robotic exoskeleton 100 can comprise one or more appendages, which in some examples can be jointed appendages, such as a right jointed ground-contacting appendage 102 and a left jointed ground-contacting appendage 104. The left and right jointed ground-contacting appendages 102 and 104 can contact the ground or other surface during locomotion to facilitate the gait-based locomotion movements, the gait-associated capabilities, and/or the stance-associated capabilities of the robotic exoskeleton 100. The appendages, such as the left and right jointed ground-contacting appendages 102 and 104 of a robot, such as the robotic exoskeleton 100, can be configured to perform movements and operations similar to those of a human foot. For example, these can be configured to operate in a manner similar to a human foot during a gait cycle or during another operation relative to a ground surface.

The left and right jointed ground-contacting appendages 102 and 104 can receive the respective feet of a user upon the user donning the robotic exoskeleton 100. Areas of the appendages of the robotic exoskeleton 100 can be referred to using common terms used to refer to parts of a human foot. The ground contacting appendage of a robot can be part of a more complex appendage in support of a body of a robot. For example, as shown, the left and right jointed ground-contacting appendages 102 and 104 can be part of a more complex appendage, namely left and right limb or leg appendages, respectfully, of the robotic exoskeleton 100 shown.

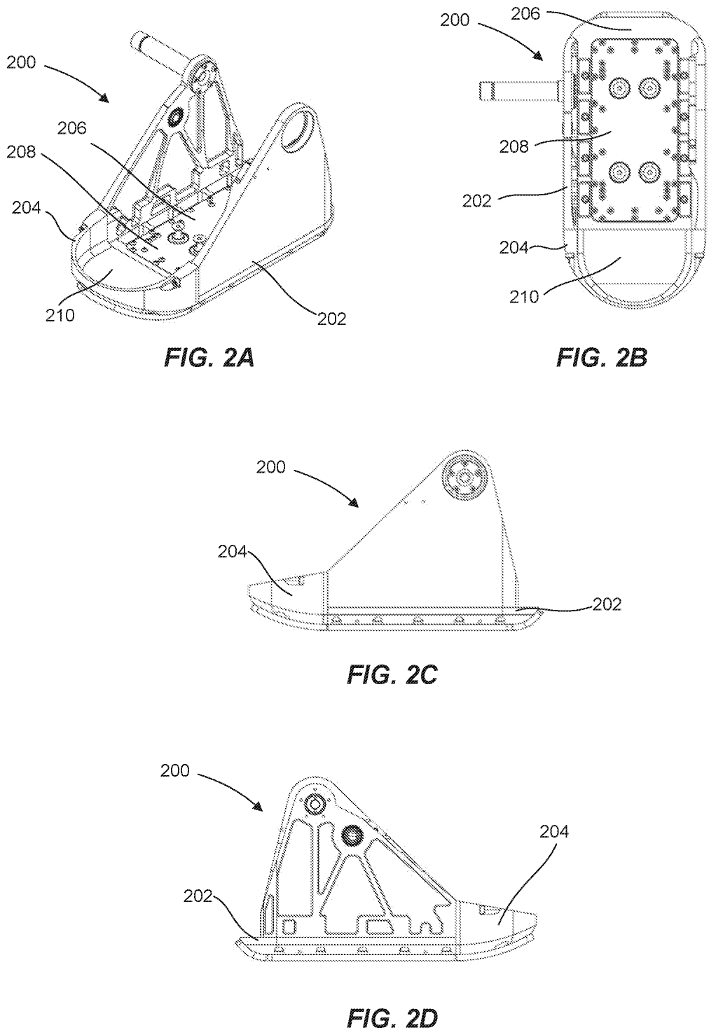

FIGS. 2A, 2B, 2C, and 2D respectively illustrate an isometric view, a top view, a left side view, and a right side view of a ground-contacting appendage operable with the robotic exoskeleton of FIG. 1, such as one of the ground-contacting appendages 102 or 104 of the robotic exoskeleton 100, which can comprise a jointed appendage part of a larger appendage of the robotic exoskeleton 100 (e.g., part of a lower limb or leg-like appendage, as shown). With reference to FIGS. 1 and 2A-2D, in this example, the ground-contacting appendage 200 can be configured to be in the form of or comprises a type operable to receive and support a foot of a human donning the wearable robotic exoskeleton 100. In one example, the ground-contacting appendage 200 can directly receive and support the foot of the user. In another example, the ground-contacting appendage 200 can indirectly support the foot of the user, such as via a type of binding system operable to releasably receive and retain a boot worn by the user. Indeed, the ground-contacting appendage, which can also in this example be referred to as a foot-supporting appendage, 200 can be configured to receive and support a foot of a user who is donning the robotic exoskeleton 100 either directly (i.e., the appendage is configured to directly releasably couple a foot (namely a shoed foot) of a user, such as via straps, buckles, or other coupling means) or indirectly (e.g., via a wearable boot and binding system (not shown)). Either direct or indirect receipt and support of a foot of a user is contemplated in the discussion below even though not specifically set forth in the discussion of any particular example. The foot-supporting appendage 200 can include a rearward support 202 (which can also be referred to as a rearward foot support as it is intended to receive and interface with a foot of a human user) configured to receive and support a hind foot portion of a foot of a user donning the robotic exoskeleton 100, upon the foot of the user being caused to interface with the foot-supporting appendage 200. The foot-supporting appendage 200 can further include a frontward support 204 (which can also be referred to as a frontward foot support as it is intended to receive and interface with a foot of a human user) configured to removably couple to the rearward support 202. The frontward support 204 can be configured to receive and support, at least in part, a forefoot portion of the foot of the user donning the robotic exoskeleton 100, the foot of the user upon the foot of the user being caused to interface with the foot-supporting appendage 200. The frontward support 204 and/or the rearward support 202 can further be configured to support a midfoot portion of the foot of the user. As such, the foot of the user can be completely supported about a support surface 206 (which can be made up of the individual support surfaces of the frontward and rearward supports) of the foot-supporting appendage 200 as the user dons and operates the wearable robotic exoskeleton 100 extending between the frontward support 204 and the rearward support 202.

The term “foot-supporting appendage” refers to a specific type of ground-contacting appendage designed to interface with a human operator or user donning and operating a wearable robot or robotic device, such as a wearable exoskeleton, namely to receive and support a foot of the user. The foot-supporting appendage can comprise various components that directly or indirectly support the foot of the user. For example, the foot-supporting appendage can be configured to directly receive and support a shoed foot of the user, such as receiving the foot of the user on a support surface, and securing the foot of the user with securing means, such as straps, buckles, etc. as will be apparent to those skilled in the art. In another example, the foot-supporting appendage can be configured to indirectly receive and support the foot of a user, such as one that is within a boot. In this example, the foot-supporting appendage can comprise a binding mechanism or intermediate interface element that is operable to receive and releasably secure the booted foot of the user to the foot-supporting appendage.

The term “frontward support” refers to a structural component of the ground-contacting appendage that, in some examples functions as the forefoot of the ground-contacting appendage without any user interfacing therewith, and in other examples, functions both as the forefoot of the ground-contacting appendage as well as the specific component of the ground-contacting appendage operable to support the forefoot of a user interfacing with the ground-contacting appendage. The type, configuration and function of the frontward support will depend upon the type of robot or robotic device in which the ground-contacting appendage is associated with, and whether or not the robot or robotic device is intended to interface with a human user. In the case of a ground-contacting appendage comprising a foot-supporting appendage type, the frontward support can be configured to support, at least in part, the forefoot of a user donning a wearable robotic exoskeleton. In this example, the frontward support can comprise a support surface oriented parallel or substantially parallel to a ground surface, and that is sized and configured to directly or indirectly receive the forefoot of the user and to support, at least in part, the weight of the user thereon. The frontward support can, in some examples, be further configured to support all or part of the midfoot of the user about the support surface.

The term “rearward support” refers to a structural component of the ground-contacting appendage that, in some examples functions as the hind foot of the ground-contacting appendage without any user interfacing therewith, and in other examples, functions both as the hind foot of the ground-contacting appendage as well as the specific component of the ground-contacting appendage operable to support the hind foot of a user interfacing with the ground-contacting appendage. The type, configuration and function of the rearward support will depend upon the type of robot or robotic device in which the ground-contacting appendage is associated with, and whether or not the robot or robotic device is intended to interface with a human user. In the case of a ground-contacting appendage comprising a foot-supporting appendage type, the rearward support can be configured to support, at least in part, the hind foot of a user donning a wearable robotic exoskeleton. In this example, the rearward support can comprise a support surface oriented parallel or substantially parallel to a ground surface, and that is sized and configured to directly or indirectly receive the hind foot of the user and to support, at least in part, the weight of the user thereon. The rearward support can, in some examples, be further configured to support all or part of the midfoot of the user about the support surface.

The term “adjustment mechanism” refers to any type of mechanism and its various components that facilitates the frontward support and the rearward support to be moveably coupled to one another, such that a plurality of different adjustment positions can be reached to adjust a size of the ground-contacting appendage of the robot or robotic system.

The wearable robotic exoskeleton 100 can further include a plurality of actuators and/or motors that work to move and control various components, appendages, and systems of the wearable robotic exoskeleton 100. The wearable robotic exoskeleton 100 itself as a whole, and/or the actuators and/or motors of the wearable robotic exoskeleton 100 can be controlled, driven, actuated, or stationary based on load measurements obtained by a load cell. A load cell 208 can be disposed on the support surface 206 and configured to directly or indirectly (e.g., via a binding system) interface with the foot of the user supported on the rearward support 202. The load cell 208 can measure interaction forces between the user and the foot-supporting appendage 200 of the wearable robotic exoskeleton 100 worn by the user. The load cell 208 can further measure ground interaction forces between the ground and the foot-supporting appendage 200 of the wearable robotic exoskeleton 100 (i.e., ground reaction forces applied by the ground on the foot-supporting appendage 200). The load cell 208 can comprise a sensor operable with a robotic joint or appendage of the wearable robotic exoskeleton 100. The load cell 208 can be a force-moment load cell that provides accurate force measurements acting on one or more orthogonal axes (±Fx, ±Fy, ±Fz) of the load cell 208 and/or one or more torques/moments (±Mx, ±My, ±Mz) acting about the one or more orthogonal axes. The load cell 208 can measure forces and/or moments present within one or more degrees of freedom. Accordingly, the load cell 208 can be a one or more degree of freedom load cell. For example, the load cell 208 can be a six degree of freedom load cell. Alternatively, multiple load cells can be strategically positioned and supported on the foot-supporting appendage 200 to accomplish the needed or desired sensing of interaction forces and to facilitate the generation of associated force measurements.

Force/torque sensors, such as load cell 208, can be used for applications where holding a steady position and trajectory of high-repeatability is necessary. The load cell 208 can allow a controller of the wearable robotic exoskeleton 100 to recognize forces and movements acting on the foot-supporting appendage 200 and improve the responsiveness and resulting motion of the wearable robotic exoskeleton 100. As a user's foot interacts with the foot-supporting appendage 200, the foot generates torque and force on the foot-supporting appendage 200, which is then measured by the load cell 208. These multi-axis torques and forces can then be measured in all the directions and the signal can be sent to the controller of the robot for analysis. The various motors and actuators of the robotic exoskeleton 100 can be driven and controlled based, at least in part, on the signal from the load cell 208.

The frontward support 204 of the foot-supporting appendage 200 can include a horizontal or lateral support surface 210 extending between walls or wall portions extending up from the lateral support surface 210. The frontward support 204 and the lateral support surface can be configured to receive and support, at least in part, the forefoot portion of the foot of a user wearing the robotic exoskeleton 100. The rearward support 202 can include a horizontal or lateral support surface 213 extending between walls or wall portions extending up from the lateral support surface 213. The rearward support 202 and the lateral support surface can be configured to receive and support, at least in part, the hind foot portion of the foot of a user wearing the robotic exoskeleton 100. The frontward support 204 can be configured to connect to the rearward support 202 to form all or a part of the foot-supporting appendage 200. As will be explained, the frontward support 204 can be removable from the rearward support 202 to facilitate adjustment of a size of the foot-supporting appendage 200. The frontward support 204 can be removed from the rearward support 202 of the foot-supporting appendage 200 and replaced with (i.e., interchanged with) another frontward support of a different size in order to change the size of the foot-supporting appendage 200. Any of a number of different sized frontward supports can be interchanged with the rearward support 202 in order to change a size of the foot-supporting appendage 200 to accommodate different users having feet of different sizes, such that the wearable robotic exoskeleton 100 can accommodate a plurality of users of a plurality of different sizes.

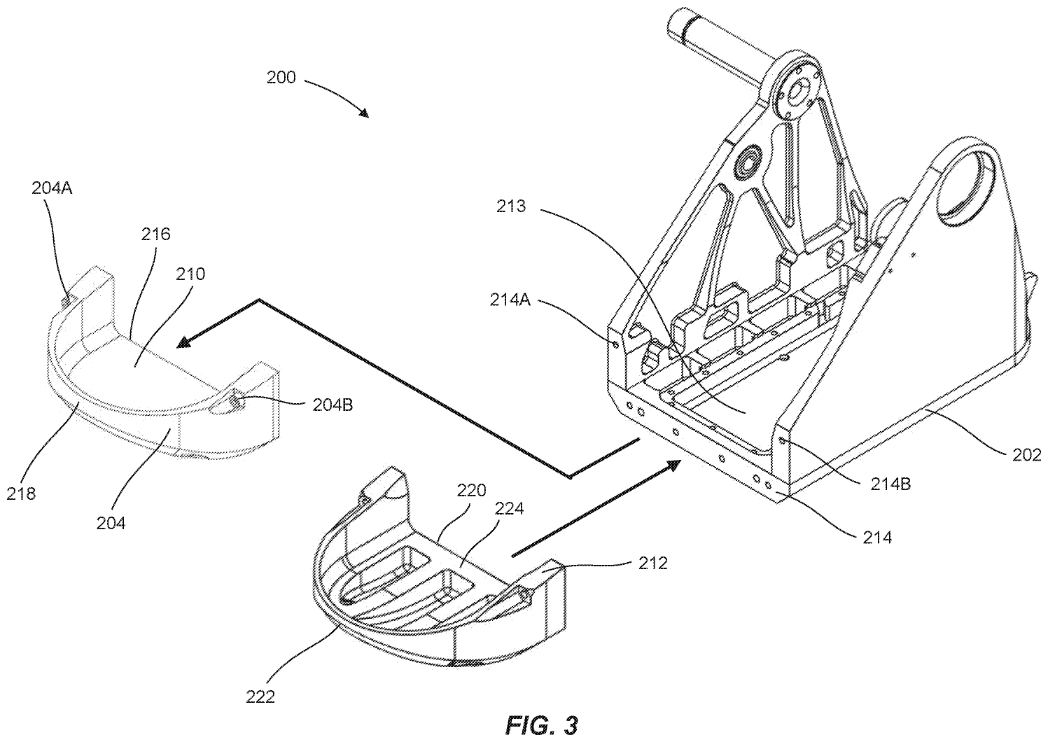

FIG. 3 illustrates an example of the foot-supporting appendage 200. In FIG. 3, the frontward support 204 is a first frontward support that is removable from the rearward support 202. The frontward support 204 can be connected to the rearward support 202 by any known attachment mechanism. For example, the frontward support 204 can include one or more fasteners 204A and 204B that extend through frontward support 204 and interface with one or more holes 214A and 214B formed in an attachment surface 214 of the rearward support 202. The mechanism of attachment of the frontward support 204 to the rearward support 202 is not intended to be limited in any way by this disclosure. For example, the frontward support 204 can be removably attached to the rearward support 202 by fasteners, screws, bolts, rods, magnets, projections, protrusions, slots, interference fittings, clips, adhesives, or any other known method or mechanism of attachment.

In FIG. 3, first frontward support 204 is originally attached to rearward support 202, and is shown in a state where the first frontward support 204 has been removed from the rearward support 202. The first frontward support 204 can have a certain size measured from a rear edge 216 configured to interface with the support surface 214 of the rearward support 202 to a front edge 218 opposite the rear edge 216. The size of the frontward support 204 can be sized to accommodate a certain size range of a foot of a user to be received in the foot-supporting appendage 200. With the first frontward support 204 removed from the rearward support 202, a second frontward support 212 can be interchanged with and attached to the rearward support 202 to form the foot-supporting appendage 200. The second frontward support 212 can be attached to the rearward support 202 by any known fastening mechanism or method as described above with reference to the first frontward support 204. For example, the frontward support 212 can be attached to the rearward support 202 by fasteners, screws, bolts, rods, magnets, projections, slots, interference fitting, clips, adhesive, or any other known method or mechanism of attachment.

The second frontward support 212 can be of a size different from the first frontward support 204. The second frontward support 212 can be different in length, width, height, or a combination of these. For illustration purposes, one size measurement of the second frontward support 212 can be a size that is measured from a rear edge 220 of the second frontward support 212 to a front edge 222 of the second frontward support 212. However, other size differences and associated size measurements are contemplated (e.g., width, height, etc.). Accordingly, removal of the first frontward support 204 and attachment of the second frontward support 212 to the rearward support 202 adjusts the size of the foot-supporting appendage 200 to accommodate a different range of sizes of feet of different users. It is also contemplated that the second frontward support 212 can alternatively be the same size as the first frontward support 204, but different in an aspect other than size that would provide cause for being interchanged with the first frontward support 204. For example, relative to the first frontward support 204, the second frontward support 204 can comprise a different material makeup, a different ground-contacting surface material (e.g., a different sole or sole component), a different support surface that interfaces with the foot of the user (e.g., rigid support surface, a compliant sole surface (e.g., memory foam), or any other different aspect.

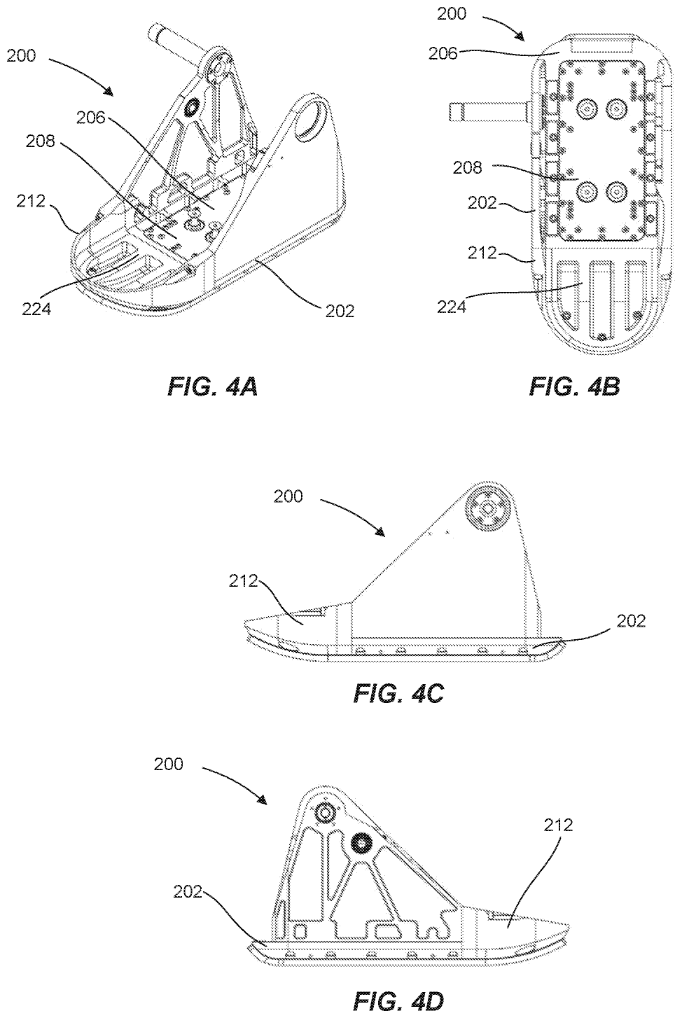

The second frontward support 212 can be configured to connect to the rearward support 202 and can further include a support surface 224 configured to receive the forefoot portion of the foot of a user wearing the robotic exoskeleton 100, with the foot interfacing with the foot-supporting appendage 200. With continued reference to FIG. 1, FIGS. 4A, 4B, 4C, and 4D respectively illustrate an isometric view, a top view, a left side view, and a right side view of the foot-supporting appendage 200 operable with the wearable robotic exoskeleton 100 in accordance with an example of the present disclosure where the foot-supporting appendage 200 includes the second frontward support 212 different from the first frontward support 204 of FIGS. 2A-2D. As shown, the foot-supporting appendage 200 includes a different sized frontward support 212 than what is shown in FIGS. 2A-2D. The frontward support 212 is shown attached to the rearward support 202 to create a foot-supporting appendage of a different size than what is shown in FIGS. 2A-2D.

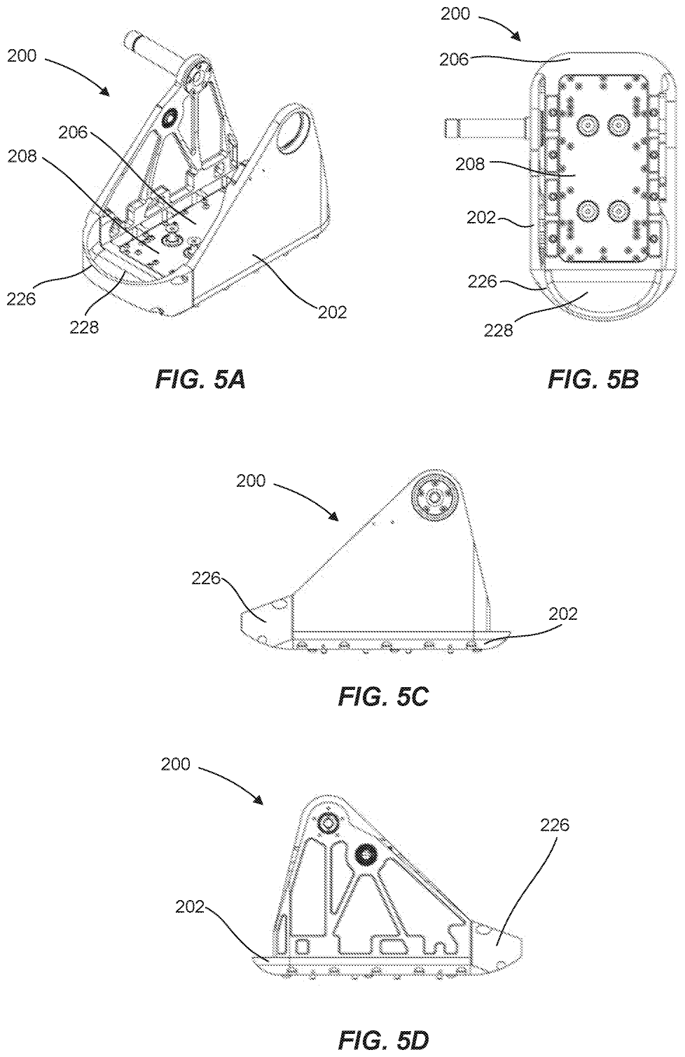

With continued reference to FIG. 1, FIGS. 5A, 5B, 5C, and 5D respectively illustrate an isometric view, a top view, a left side view, and a right side view of a foot-supporting appendage 200 of a wearable robotic exoskeleton in accordance with an example of the present disclosure where the foot-supporting appendage 200 includes a third frontward support 226 different from the first frontward support 204 and the second frontward support 212. As shown, the foot-supporting appendage includes a different sized frontward support 226 than what is shown in FIGS. 2A-2D and FIGS. 4A-4D. The frontward support 226 is shown attached to the rearward support 202 to create a foot-supporting appendage 200 of a different size than is the frontward supports shown in FIGS. 2A-2D and FIGS. 4A-4D. The frontward support 226 can be configured to connect to the rearward support 202 and can further include a support surface 228 configured to receive the forefoot portion of the foot of a user wearing the exoskeleton, with the foot interfacing with the foot-supporting appendage 200.

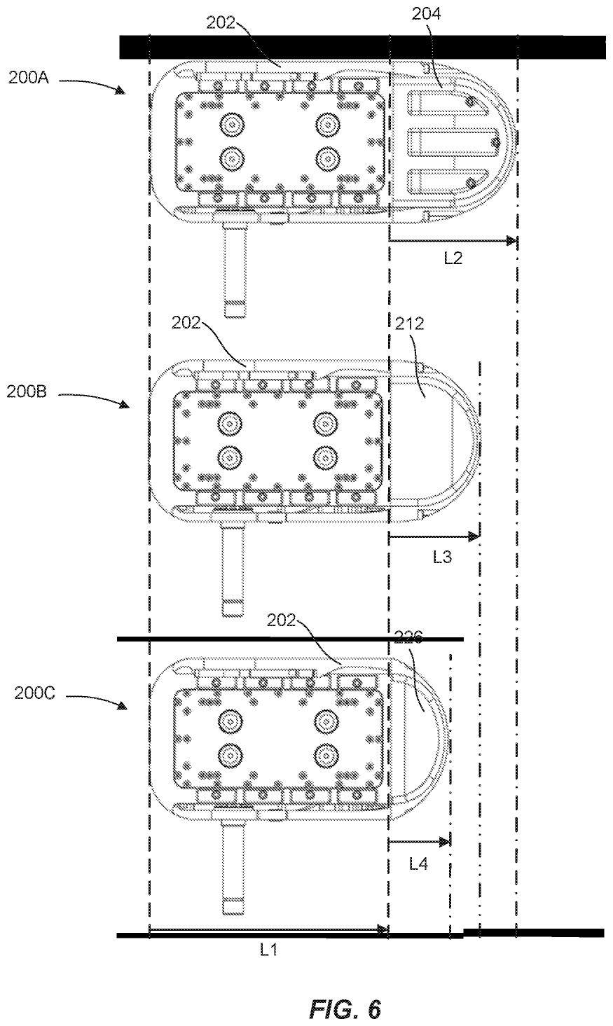

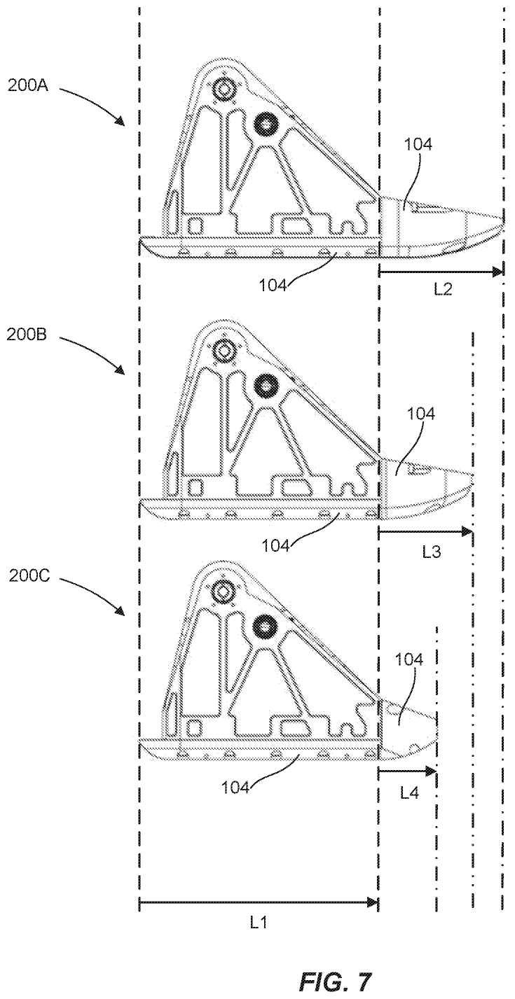

A top-view comparison of the foot-supporting appendage 200 with the various frontward supports 204, 212, and 226 attached to rearward support 202 is shown in FIG. 6. A side-view comparison of the foot-supporting appendage 200 with the various frontward supports 204, 212, and 226 attached to rearward support 202 is shown in FIG. 7. It is to be understood that the three views shown are alternate configurations of the same foot-supporting appendage 200, just with the different frontward supports 204, 212 and 226 attached. However, for purposes of clarity, the three configurations will be identified as foot-supporting appendage configurations 200A, 200B, and 200C. As shown, the rearward support 202 of each foot-supporting appendage configuration 200A, 200B, and 200C can be the same and can have a length of L1 measured from a rear edge to a front edge of the rearward support 202. The length L1 can be any length needed or desired.

Configuration 200A illustrates a configuration of the foot-supporting appendage 200 where the rearward support 202 is attached to the first frontward support 204. The first frontward support 202, as illustrated, can have a length of L2 measured from a rear edge to a front edge of the frontward support 204. Configuration 200B illustrates a configuration of the foot-supporting appendage 200 where the rearward support 202 is attached to the second frontward support 212. The second frontward support 212, as illustrated, can have a length of L3 measured from a rear edge to a front edge of the frontward support 212. Configuration 200C illustrates a configuration of the foot-supporting appendage 200 where the rearward support 202 is attached to the third frontward support 226. The third frontward support 226, as illustrated, can have a length of L4 measured from a rear edge to a front edge of the frontward support 226.

As shown, the length L2 of frontward support 204 can be longer than length L3 of frontward support 212 and length L4 of frontward support 226. Furthermore, the length L3 of frontward support 212 can be longer than length L4 of frontward support 226 and shorter than length L2 of frontward support 202. The length L4 of frontward support 226 can be shorter than length L2 of frontward support 202 and shorter than length L3 of frontward support 212. Accordingly, to support feet of different sizes for different users who may don the wearable robotic exoskeleton 100, the frontward support can be selected from any of frontward supports 202, 212, and 226 to fit and support a foot of a particular user wearing the exoskeleton.

It will be understood that more frontward supports than those illustrated can be used to fit different feet of different users. For example, while only three frontward supports 202, 212, and 226 are illustrated, additional frontward supports having other sizes can also be used, such as frontward supports that have lengths shorter than the length L4 of the frontward support 226 that are configured to support smaller sized feet on the foot-supporting appendage 200. Additionally, frontward supports having lengths larger than the length L2 of the frontward support 202 can be used to support larger sized feet on the foot-supporting appendage 200. Additionally, frontward supports of sizes anywhere between and including frontward supports 204 and 226 can be attached to rearward support 202 in order to accommodate any foot sizes on the foot-supporting appendage 200.

As illustrated, rearward support 202 can be the same size and length L1 in all configurations 200A, 200B, and 200C of the foot-supporting appendage 200. It has been observed that with human feet of different sizes, the greatest differences in sizes occurs in portions of the feet that are forward of the ankle without much difference in distance between the heel and ankle of the different sized feet. In other words, when comparing a foot of a large size to a foot of a smaller size, the comparable distance between the ankle and the heel is typically much less than the comparable distance between the ankle and the ends of the toes. Indeed, a large difference is observed in the length between the ankle and the end of the toes between a larger sized foot and a smaller sized foot. Due to the observed differences between larger sized feet and smaller sized feet, the size of a foot-supporting appendage 200 can be adjusted and resized for a plurality of different sizes of feet by only altering the portion of the foot-supporting appendage in support of the forefoot of various users, which in this example, comprises interchanging the frontward supports 204, 212, and 226 of the foot-supporting appendage 200 to achieve the different foot-supporting appendage configurations 200A, 200B, and 200C. The rearward support 202 can be the same size and, in many cases, does not need to be replaced in order to adjust a size of a foot-supporting appendage 200. This configuration of resizing a foot-supporting appendage 200 leads to an advantageous configuration where only a small portion (the frontward support) of the foot-supporting appendage 200 needs be removed and replaced to adjust a wearable robotic exoskeleton to accommodate the sizes of multiple users quickly and efficiently. Additionally, the manufacturing of different sized frontward supports is much easier than creating full foot-supporting appendages of different sizes for different users of the robotic exoskeleton 100.

The number and configuration of interchangeable frontward supports can vary, and can be made to accommodate sizes of feet in any desired or needed range. In one example, which is not intended to be limiting in any way, the three different frontward supports 202, 212, and 226 illustrated in FIGS. 1-7 can be configured and sized to accommodate shoe sizes ranging from a size 5 female shoe to a size 13 male shoe in American shoe sizing standards. It will be appreciated that scaling parts of the foot-supporting appendage 200 to be larger or smaller can allow for creating a foot-supporting appendage of any size to fit any foot of any user.

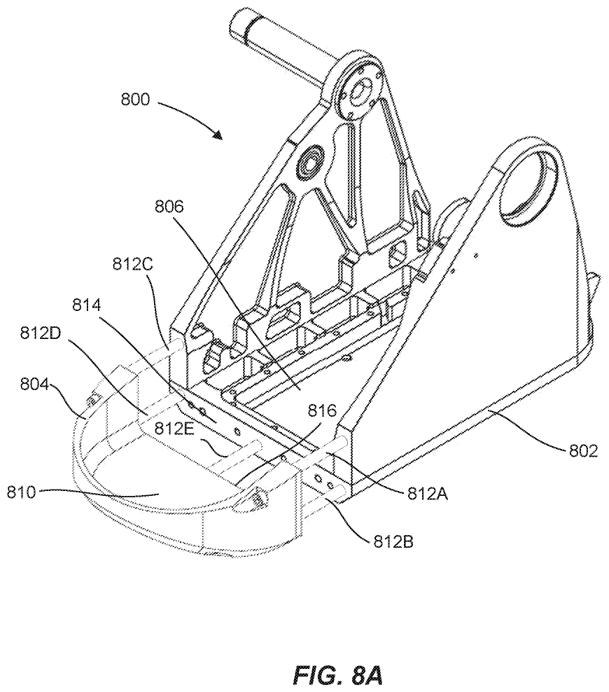

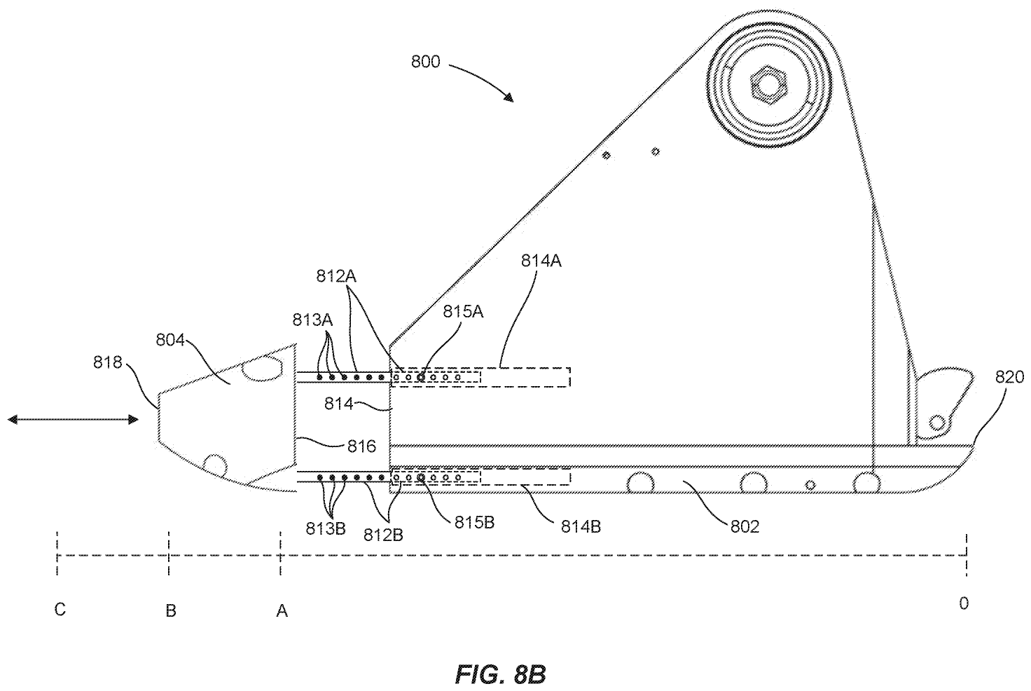

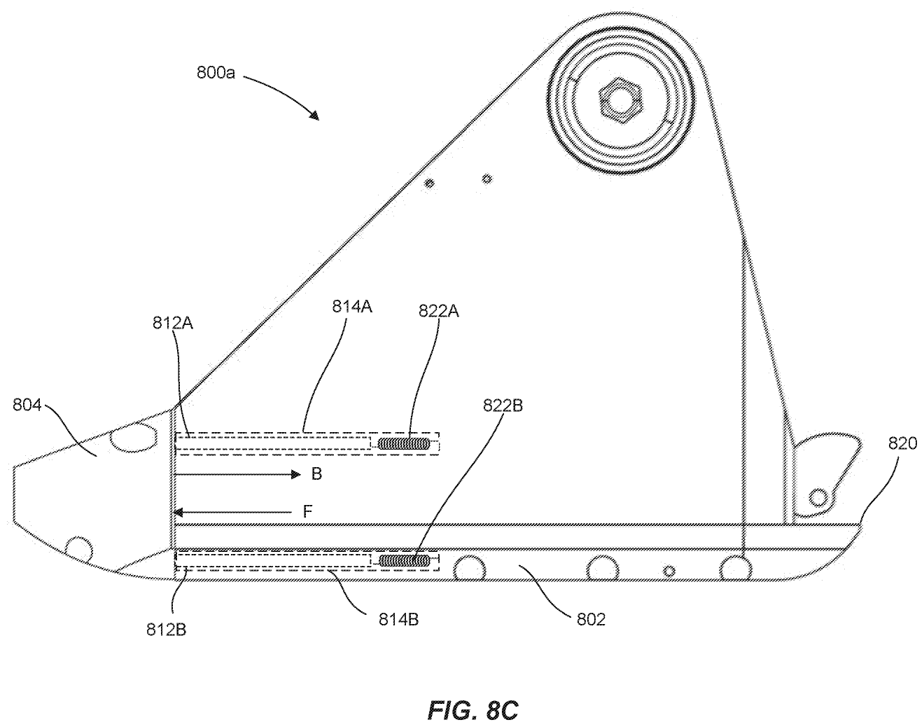

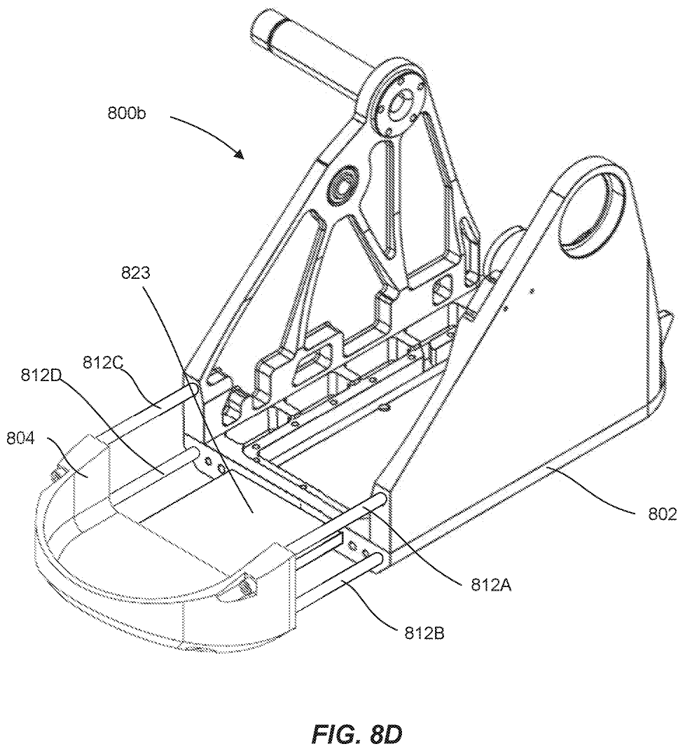

Alternative configurations for sizing and adjusting a foot-supporting appendage of a wearable robotic exoskeleton are also contemplated and described herein. Indeed, FIGS. 8A-11 illustrate the frontward support being configured to be extendable and retractable relative to the rearward support of the respective foot-supporting appendages as part of an adjustment mechanism that facilitates the sizing and adjustability of the respective foot-supporting appendages. Each of these are discussed in detail below.

With continued reference to FIG. 1, FIGS. 8A and 8B illustrate a foot-supporting appendage 800 of the robotic exoskeleton 100 according to an example of the present disclosure. As illustrated in FIG. 8A, the foot-supporting appendage 800 can include a rearward support 802 configured to support a hind foot portion of a foot of a user donning the robotic exoskeleton 100, with the foot of the user interfacing with the foot-supporting appendage 800. The foot-supporting appendage 800 can further include a frontward support 804 configured to connect to the rearward support 802. The frontward support 804 can be configured to support a forefoot portion of the foot of the user. The rearward support 802 can be configured to support the hind foot of the user on a support surface 806 thereof that is configured to directly or indirectly receive the foot of the user. The frontward support 804 can be comprise and be configured with a support surface 810 operable to receive and support the forefoot of the user.

In this example, the frontward support is configured to be extendable and retractable relative to the rearward support as part of an adjustment mechanism that facilitates the sizing and adjustability of the foot-supporting appendage 800. The frontward support 804 can be configured to movably couple (i.e., joined or coupled in a manner that facilitates moving of the frontward support 804 relative to the rearward support 802) to the rearward support 802 via an adjustment mechanism. In the example shown, the adjustment mechanism can comprise the frontward support 804 slidably attached to the rearward support 802 via one or more translational support rods, such as the illustrated translational support rods 812A, 812B, 812C, 812D, and 812E, wherein the “slidable attachment” represents only one example type of movement of the frontward support 804 relative to the rearward support 802. The support rods 812A-812E can be configured to interface with and extend from an interface surface 814 of the rearward support 802 and an interface surface 816 of the frontward support 804. At least one of the frontward support 804 or the rearward support 802 can be slideably engaged with one or more of the support rods 812A-812E to facilitate lengthening and/or shortening the foot-supporting appendage 800. In the example shown, this can be accomplished by actuating (e.g., moving or sliding) at least one of the frontward support 804 or the rearward support 802 relative to the other on the support rods 812 to adjust the size of the foot-supporting appendage 800. Actuating, moving, or sliding of the frontward support 804 or the rearward support 802 relative to the other on the support rods 812 can be accomplished by manual manipulation by a user of the supports 804 or 802 on the support rods 812 or by motorized or mechanical actuation of the supports 804 or 802 on the support rods 812. As illustrated in FIGS. 8A and 8B, the translational support rods 812 can be attached to the interface surface 816 of the frontward support 804 and can be slideably disposed in cavities formed in the rearward support 802. One or more stoppers or fasteners can be used to secure the support rods 812 in place, and to prevent the support rods 812 from inadvertently detaching or uncoupling from the frontward support 804 or the rearward support 802.

FIG. 8B illustrates an example of extending and retracting frontward support 804 relative to rearward support 802 to lengthen or shorten the length of the foot-supporting appendage 800. In the example shown, the support rods 812 can be fixedly attached or integrally formed with the frontward support 804 to extend from the interface surface 816 of the frontward support 804. The rearward support 802 can include one or more cavities 814 formed in and through the interface surface 814 of the rearward support 802. The cavities 814 can be configured to align with the support rods and to receive the support rods therein. In the side view shown, the support rods 812A and 812B can slideably engage with the cavities 814A and 814B, respectively. The support rods 812A and 812B can be actuated to slide inward and outward within cavities 814A and 814B to adjust a length of the foot-supporting appendage 800 measured from a rear edge 820 of the rearward support 802 to a front edge 818 of the frontward support 804. Although not shown in this view, the remaining support rods 812C-812E shown in FIG. 8A can be configured and can function in a similar manner to slideably engage with additional respective cavities in the rearward support 802.

The support rods 812A and 812B can be slid completely within the cavities 814A and 814B until the interface surface 816 of the frontward support 804 is disposed adjacent to and abuts or seats against the interface surface 814 of the rearward support 802. In this configuration, the foot-supporting appendage 800 has a length of A measured from the rear edge 820 of the rearward support 802 to the front edge 818 of the frontward support 804. The support rods 812A and 812B can be slid nearly completely out of the cavities 814A and 814B until the interface surface 816 of the frontward support 804 is disposed at a maximum possible distance from the interface surface 814 of the rearward support 802. A stopping mechanism can prevent the rods from completely exiting the cavities 814A and 814B and separating from the rearward support 802. For example, a protrusion at the end of the support rods 812 can engage with a structural portion of the rearward support 802 adjacent of the cavities 814A and 814B to prevent the support rods 812A and 812B from being removed from the cavities 814A and 814B. In the maximum extended configuration, the foot-supporting appendage 800 has a length of C measured from the rear edge 820 of the rearward support 802 to the front edge 818 of the frontward support 804. Furthermore, the foot-supporting appendage 800 can be adjusted to any length in between length A and length C, such as length B, for example.

Furthermore, the support rods 812A and 812B can be held in place at any desired length in order to firmly size the foot-supporting appendage 800 to a proper size for a certain user donning the wearable robotic exoskeleton comprising the foot-supporting appendage 800. The support rods 812 can be held in place at a desired position by interference fitting, or by friction fitting within the cavities 814A and 814B, such that friction resists extension and retraction of the support rods 812 within the cavities 814 (and extension and retraction of the forward support 804 relative to the rearward support 802) unless a sufficient force is applied to overcome the friction between the support rods 812 and the cavities 814A.

Alternatively, locks, clamps, or other locking mechanisms can be provided to lock the frontward support 804 and support rods 812 relative to the rearward support 802 at a desired position and size for the foot-supporting appendage 800 to accommodate a foot of a user. For example, as shown in FIG. 8B, a locking mechanism can comprise support rods 812A and 812B having a plurality of holes 813A and 813B formed there through along a length of the support rods 812A and 812B. Each of the plurality of holes 813A and 813B can be made to correspond with through holes 815A and 815B formed in the rearward support 802 as the frontward support 804 and the support rods 812 slide with respect to the rearward support 802. A pin or fastener can be inserted through a desired one of the through holes 815A and 815B and corresponding holes 813A and 813B on the support rods 812A and 812B upon the proper position of the frontward support 804 relative to the rearward support 802 being achieved to fix the frontward support 804 in a desired position relative to the rearward support 802. Of course, other types of locking mechanisms can be used to achieve the same function of locking the frontward support 804 relative to the rearward support 802 at a desired position and size for the foot-supporting appendage 800 to accommodate a foot of a user, which will be apparent to those skilled in the art upon reading the present disclosure.

FIG. 8C illustrates an alternative foot supporting appendage 800a. It will be appreciated by those skilled in the art that the modifications shown in FIG. 8C can be applied to other exemplary configuration shown and described in this disclosure. As illustrated in FIG. 8C, the foot supporting appendage 800a can include the frontward support 804 coupled to the rearward support 802. The frontward support 804 can be coupled to the rearward support 802 using one or more biasing members 822. For example, the support rods 812A and 812B coupled to the frontward support 804 can be disposed in the cavities 814A and 814B and coupled to the rearward support 802 within the cavities 814A and 814B by the biasing members 822A and 822B. The biasing members 822 connecting the frontward support 804 to the rearward support 802 can apply a biasing force to the frontward support 804 to bias the frontward support 804 to rest against the rearward support 802. In other words, the frontward support 804 can be biased to a smallest possible size of the adjustable foot supporting appendage 800a. In this configuration, a user can apply a frontward force F to the frontward support 804 to overcome a biasing force B applied by the biasing members 822. The force F can be applied, for example, by the toe of a user engaging with the frontward support 804 when placing a foot into the foot supporting appendage 800a, thereby expanding the foot supporting appendage 800a to the proper size for the user. The user can then engage a heel with the rearward support 802 to engage a foot with the foot supporting appendage 800a. In such a configuration, the act of a user putting a foot in the foot supporting appendage 800a can automatically size the foot supporting appendage 800a to a proper size to accommodate a foot of the user. The biasing force B applied by the biasing members 822 can act to maintain a secure fit of the foot supporting appendage 800a on the foot of the user.

As described above with reference to other examples, the foot supporting appendage 800a can further include a lock (e.g, locks, clamps, or other locking mechanisms described herein) to secure the foot supporting appendage 800a at the proper size for the user. The lock could be either operator-engaged or automatic. Following use of the foot supporting appendage 800a by the user, the user can remove the foot from the foot supporting appendage 800a. The lock mechanism can then be released either automatically or by the user after the user exited the foot supporting appendage 800a. By release of the lock, the biasing force B applied by the biasing members can cause the foot supporting appendage 800a to return to the smallest size (e.g., where the frontward support 804 rests against the rearward support 802) to allow for sizing for the next user. Alternatively, if the same user is going to use foot supporting appendage 800a in a subsequent operation, then the lock can be left in the locked location to maintain the foot supporting appendage 800a at the desired size for the user.

The biasing members 822, as shown, can be a coil spring. Of course, other types of biasing mechanisms can be used to achieve the same function of biasing the frontward support 804 toward the rearward support 802, which will be apparent to those skilled in the art upon reading the present disclosure. For example, pneumatic, electro-mechanical, magnetic, or other biasing mechanisms can also perform the function of the biasing members 822.

FIG. 8C illustrates an alternative foot supporting appendage 800b. It will be appreciated by those skilled in the art that the modifications shown in FIG. 8D can be applied to other exemplary configuration shown and described in this disclosure. As illustrated in FIG. 8C, the foot supporting appendage 800a can include the frontward support 804 coupled to the rearward support 802 using support rods 812A, 812B, 812C, and 812D. As shown in FIG. 8D, the support rod 812E need not be rod shaped but can instead be a slidable support plate 823 attached to the frontward support 804 and slidably engaged with the rearward support 802. Similar to support rods 812A-812E shown in FIGS. 8A and 8B, the support plate 823 can engage with the rearward support 802 by sliding into a cavity formed in the rearward support 802. As the frontward support 804 is extended or retracted relative to the rearward support 802, the support plate 823 can slide outward from the cavity in rearward support 802. The support plate 823 provides an advantage over the support rod 812E in that the support plate provides a sole support surface for a foot of the user to engage with while operating the robotic exoskeleton. In other words, instead of having a gap between the frontward support 804 and the rearward support 802, a support surface is provided to better support the foot of the user. Other support plates similar to support plate 823 can be included in any of the exemplary foot supporting appendages described herein to provide a sole support surface in a gap between the frontward support and rearward support.

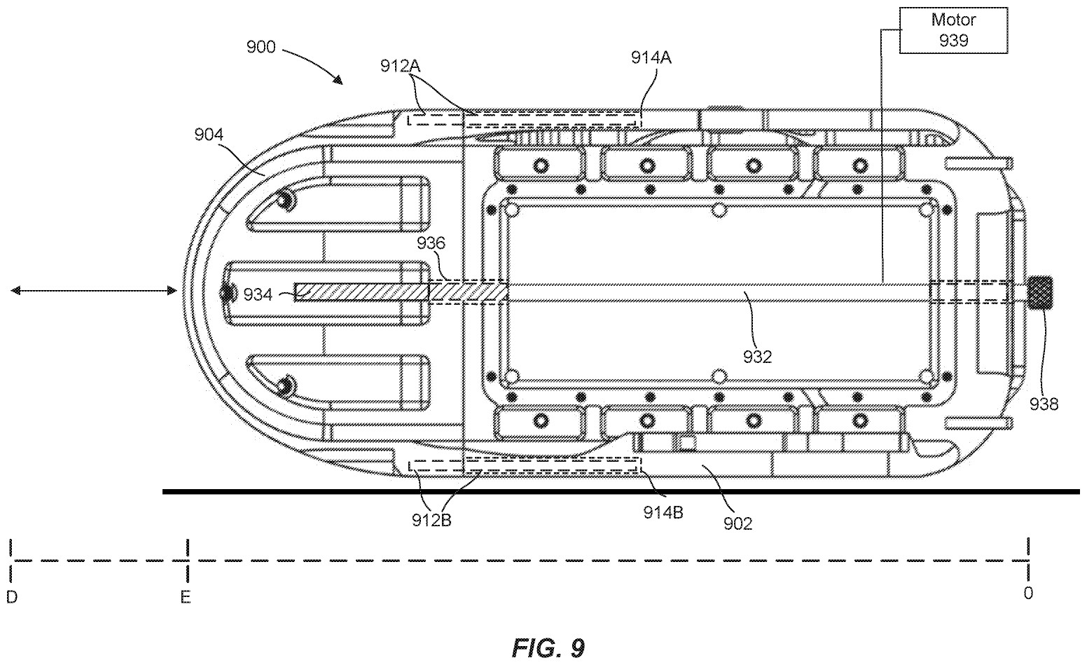

With continued reference to FIG. 1, FIG. 9 illustrates a foot-supporting appendage 900 in accordance with an example of the present disclosure. As illustrated in FIG. 9, foot-supporting appendage 900 can include a rearward support 902 configured to support a hind foot portion of a foot of a user donning the robotic exoskeleton 100, with the foot of the user interfacing with the foot-supporting appendage 900. The foot-supporting appendage 900 can further include a frontward support 904 configured to connect to the rearward support 902. The frontward support 904 can be configured to support a forefoot portion of a foot of a user donning the robotic exoskeleton 100. The rearward support 902 can be configured to support the hind foot of the user on a support surface 906 thereof that is configured to directly or indirectly receive the foot of the user. The frontward support 904 can be configured to support the forefoot of the user on a support surface 910 thereof that is configured to directly or indirectly receive the foot of the user.

The frontward support 904 can be configured to movably couple to the rearward support 902 via an adjustment mechanism. In the example shown, the adjustment mechanism can comprise the frontward support 904 slidably attached to the rearward support 902 via one or more translational support rods, such as the illustrated support rods 912A and 9128. The support rods 912 can be configured to interface with the rearward support 902 and the frontward support 904. As illustrated, the support rods 912 can be attached to one or more of the rearward support 902 and the frontward support 904. The support rods 912 can be slideably disposed within a respective cavity of the other of the rearward support 902 or the frontward support 904 (e.g., see cavities 914A or 9148 formed in the rearward support 902), and can be configured to slide in and out of the cavities. In other words, either one of the frontward support 904 or the rearward support 902 can be slideably engaged with the support rods 912 to facilitate lengthening and/or shortening of the foot-supporting appendage 900 by moving the frontward support 904 and the rearward support 902 away from each other or toward each other on the support rods 912 to adjust the size of the foot-supporting appendage 900.

Additionally, or alternatively, the rearward support 902 and the frontward support 904 can be connected through, and the adjustment mechanism can further comprise, one or more adjustment rods 932 that extends from the rearward support 902 to the frontward support 904. The adjustment rod 932 can include a threaded end 934 that interfaces with a threaded hole 936 formed in the frontward support 904. The adjustment rod 932 can be coupled to rearward support 902 to prevent sliding movement and can be freely rotatable within the rearward support 902 by manual rotation of an adjustment knob 938 attached to an end of the adjustment rod 932, or by electrical rotation of the adjustment rod 932 via a motor and any needed drivetrain assembly 939. Rotation of the knob 938 or actuation of the motor 939 can rotate the adjustment rod 932, thereby causing the threaded end 934 to rotate within the threaded hole 936 of the frontward support 904 and the frontward support 904 to either extend or retract toward or away from the rearward support 902 depending upon the direction the adjustment rod 932 is rotated.

It will be appreciated that, while the adjustment rod 932 interfaces with a threaded hole in the frontward support 904, an alternative configuration can be made where the adjustment rod extends from the frontward support 904 and has a threaded end that interfaces with a threaded hole in the rearward support 902, with the adjustment knob attached to an end of the adjustment rod at the frontward support 904.

The extension/retraction of the frontward support 904 can be supported and stabilized by the support rods 912 interfaced with both the frontward support 904 and the rearward support 902. By rotation of the adjustment rod 932, the frontward support 904 can be moved away from or toward the rearward support 902 in order to size the foot-supporting appendage 900 to any length between length E and length D shown in FIG. 9 as measured from the back of the foot-supporting appendage 900. As such, it will be appreciated that scaling parts of the foot-supporting appendage 900 to be larger or smaller can allow for the foot-supporting appendage 900 to be adjustable to different sized feet of different users.

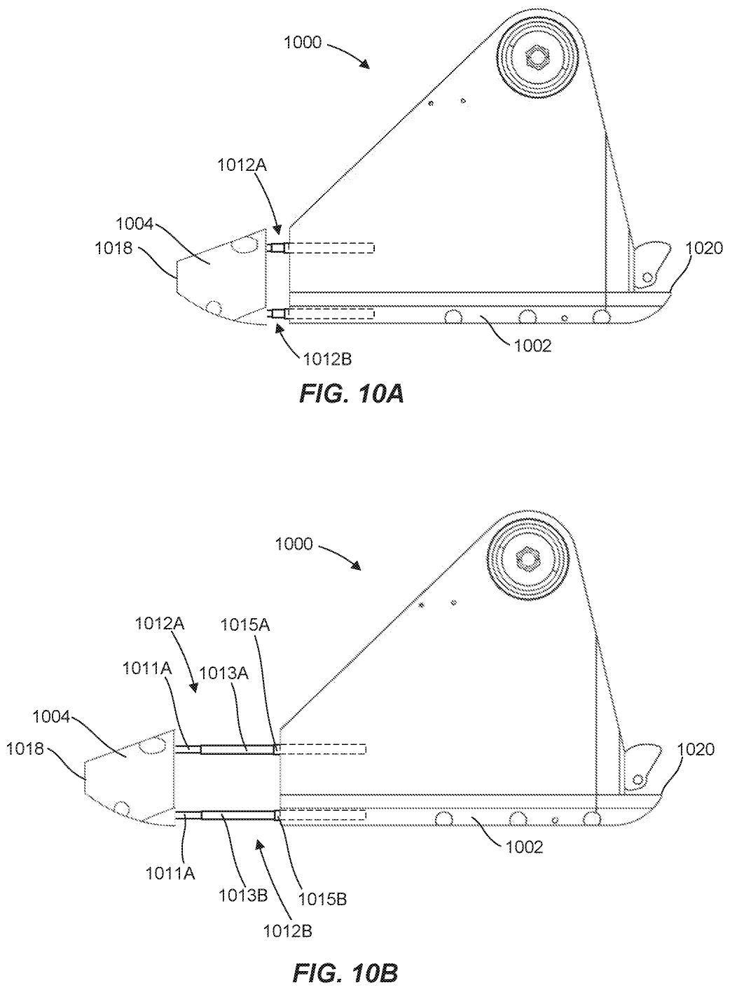

With continued reference to FIG. 1, FIGS. 10A and 10B illustrate a foot-supporting appendage 1000 in accordance with an example of the present disclosure. As illustrated in FIG. 10A, foot-supporting appendage 1000 can include a rearward support 1002 configured to support a hind foot portion of a foot of a user donning the robotic exoskeleton 100, with the foot of the user interfacing with the foot-supporting appendage 1000. The foot-supporting appendage 1000 can further include a frontward support 1004 configured to connect to the rearward support 1002. The frontward support 1004 can be configured to support a forefoot portion of a foot of a user donning the robotic exoskeleton 100. The rearward support 1002 can be configured to support the hind foot of the user on a support surface 1006 thereof that is configured to directly or indirectly receive the foot of the user. The frontward support 1004 can be configured to support the forefoot of the user on a support surface 1010 thereof that is configured to directly or indirectly receive the foot of the user.

The frontward support 1004 can be configured to moveably couple to the rearward support 1002 via an adjustment mechanism. In the example shown, the adjustment mechanism can comprise the frontward support 1004 slidably attached to the rearward support 1002 via one or more telescoping rods 1012, such as the illustrated telescoping rods 1012A and 1012B. The telescoping rods 1012 can be configured to interface with the rearward support 1002 and the frontward support 1004. As illustrated, the telescoping rods 1012 can be attached to the rearward support 1002 and the frontward support 1004. The telescoping rods 1012 can be disposed within the rearward support 1002 and the frontward support 1004. In other words, the frontward support 1004 and the rearward support 1002 can be engaged with the telescoping rods 1012 to facilitate lengthening and/or shortening the foot-supporting appendage 1000 by extending or retracting the telescoping rods 1012 to move the frontward support 904 and the rearward support 902 away from each other or together to adjust the size of the foot-supporting appendage 1000.

As shown in FIG. 10B, the telescoping rods 1012A and 1012B can each include a plurality of rod segments 1011A, 1013A, 1015A, and 1011B, 1013B, 1015B. The segments can be nested together within each other in order to allow for extension and retraction of the frontward support 1004 away from the rearward support 1002 by actuation (e.g., extension and retraction) of the telescoping rods. A portion of each of the telescoping rods 1012 can be secured within respective cavities formed in one of the rearward support 1002 or the frontward support 1004 (e.g., see the cavities formed in the rearward support 1002 in FIGS. 10A and 10B). Opposite ends of the respective telescoping rods 1012 can be secured to the other of the rearward support 1002 or the frontward support 1004. These opposite ends can be inserted into and secured within respective cavities formed in the other of the rearward support 1002 or the frontward support 1004, or they can be secured to the interface surface (e.g., see interface surfaces 214 and 220 of FIG. 3) of the other of the rearward support 1002 or the frontward support 1004, such as via a flange supported about the ends of the telescoping rods 1012, with the flange secured to the interface surface via fasteners.

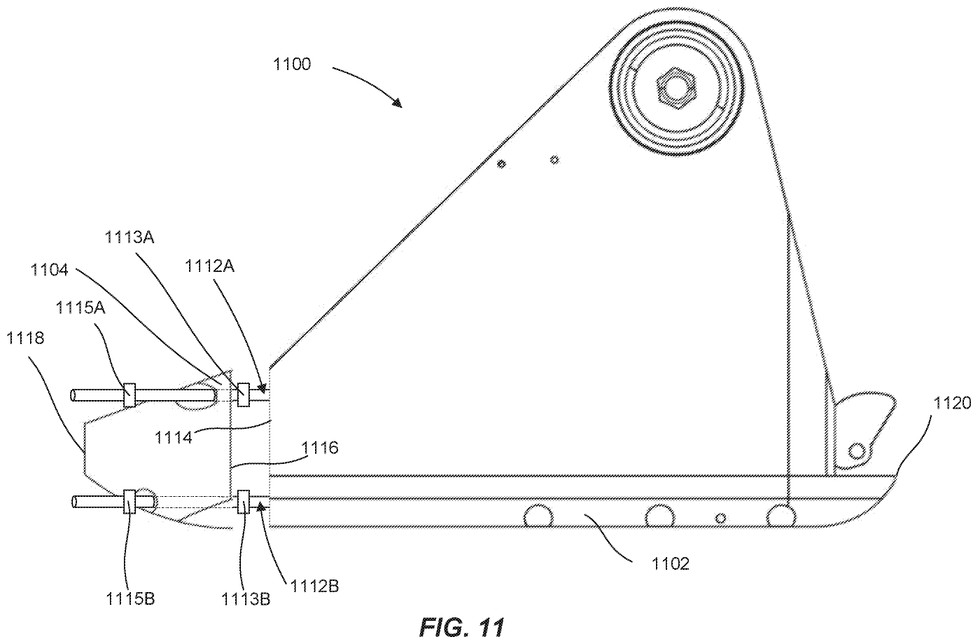

With continued reference to FIG. 1, FIG. 11 illustrates a foot-supporting appendage 1100 in accordance with an example of the present disclosure. As illustrated in FIG. 11, foot-supporting appendage 1100 can include a rearward support 1102 configured to support a hind foot portion of a foot of a user donning the robotic exoskeleton 100, with the foot of the user interfacing with the foot-supporting appendage 1100. The foot-supporting appendage 1100 can further include a frontward support 1104 configured to connect to the rearward support 1102. The frontward support 1104 can be configured to support a forefoot portion of a foot of the user. The rearward support 1102 can be configured to support the hind foot of the user on a support surface 1106 thereof that is configured to directly or indirectly receive the foot of the user. The frontward support 1104 can be configured to support the forefoot of the user on a support surface 1110 thereof that is configured to directly or indirectly receive the foot of the user.

The frontward support 1104 can be configured to movably couple to the rearward support 1102 via an adjustment mechanism. In the example shown, the adjustment mechanism can comprise the frontward support 1104 slidably attached to the rearward support 1102 via one or more support rails 1112 (which can be located similarly to support rails 812A, 8128, 812C, 812D, and 812E shown in FIG. 8A), such as the illustrated support rails 1112A and 1112B. The support rails 1112 can be configured to interface with an interface surface 1114 of the rearward support 1102 and an interface surface 1116 of the frontward support 1104. Either of the frontward support 1104 or the rearward support 1102 can be slideably engaged with the support rails 1112 to facilitate lengthening and/or shortening of the foot-supporting appendage 1100 by moving the frontward support 1104 and the rearward support 1102 away from each other or toward each other on the support rails 1112 to adjust the size of the foot-supporting appendage 1100. For example, as illustrated in FIG. 11, the support rails 1112 can be attached to the rearward support 1102 and configured to extend from the interface surface 1114 of the rearward support 1102. In this example, the support rails 1112 can be slideably disposed through the frontward support 1104.

FIG. 11 illustrates an example of extending and retracting the frontward support 1104 relative to the rearward support 1102 to lengthen or shorten the length of the foot-supporting appendage 1100. As illustrated, the support rails 1112 can be attached to the rearward support 1102 to extend from the interface surface 1114 of the rearward support 1104. In the example shown, the support rails 1112 can be fixedly attached or integrally formed with the rearward support 1102. The frontward support 1104 can be slid on the support rails 1112 until the interface surface 1116 of the frontward support 1104 is disposed adjacent to and abuts against the interface surface 1114 of the rearward support 1102.

Furthermore, the frontward support 1104 can be moved on the support rails 1112A and 1112B and can be held in place at any desired length on the support rails 1112A and 112B in order to firmly size the foot-supporting appendage 1100 to a proper size for a certain user donning the wearable robotic exoskeleton 100. The frontward support 1104 can be held on the support rails 1112 by interference fitting, or by friction fitting between the support rails 1112 and the frontward support 1104. For example, the friction resists extension and retraction of the frontward support 1104 on the support rails 1112A and 1112B unless a sufficient force is applied to overcome the friction between the support rails 1112A and 1112B and the frontward support 1104.

Locks, clamps, or other locking mechanisms can be provided to lock the frontward support 1104 on the support rails 1112 at a desired position to achieve a desired size for the foot-supporting appendage 1100 to accommodate a foot of a user. For example, as shown in FIG. 11, a locking mechanism can comprise one or more of the support rails 1112 including a first locking clamp and a second locking clamp. The support rail 1112A can include a first locking clamp 1113A, disposed on the support rail 1112A at a position between the rearward support 1102 and the frontward support 1104, and a second locking clamp 1115A disposed on an opposite side of the frontward support than the first locking mechanism 1113A. The support rail 1112B can include a first locking clamp 1113B, disposed on the support rail 1112B at a position between the rearward support 1102 and the frontward support 1104, and a second locking clamp 1115B disposed on an opposite side of the frontward support than the first locking mechanism 1113B. The locking clamps 1113A, 1113B, 1115A, and 1115B can be configured to fixedly clamp to the support rails at an infinite number of positions, thereby setting a range in which the frontward support 1104 is able to slide on the support rails 1112 before abutting the locking mechanisms 1113A, 1113B, 1115A, and 1115B.

In another example, the locking mechanism can comprise support rails 1112 that comprise threaded rods and one or more locking nuts 1113A, 1113B, 1115A, and 1115B, which can be configured as threaded nuts that mate with the threads on the support rails 1112. In the threaded configuration, the locking nuts 1113A, 1113B, 1115A, and 1115B can be rotated on the threaded support rails 1112 to abut with the frontward support supported on the support rails 1112 in a desired position. The first locking nuts 1113A and 1113B can be rotated to abut a rear of the frontward support 1104 to set a rear stop at which the frontward support 1104 is prevented from moving any further toward the rearward support 1102. The second locking nuts 1115A and 1115B can be rotated on the threaded rods to abut a front portion of the frontward support 1104, thereby setting a front stop at which the frontward support 1104 is prevented from moving any further away from the rearward support 1102. The locking nuts 1113A, 1113B, 1115A, and 1115B can each abut the frontward support 1104 to pinch or clamp the frontward support 1104 firmly between the locking nuts in a desired position on the support rails 1112, thereby preventing any movement of the frontward support 1104 on the support rails 1112. Of course, other types of locking mechanism are possible and contemplated herein, which will be apparent to those skilled in the art upon reading the present disclosure.

It will be understood that while the FIG. 11 illustrates the support rails fixedly attached to the rearward support 1102 and slideably engaged with the frontward support 1104, an alternative configuration is possible where the support rails are attached to the frontward support 1104 and slideably engaged with the rearward support 1102.

Methods for modifying a size of a foot-supporting appendage of a robot or robotic device, such as a wearable robotic exoskeleton, are disclosed herein. The foot-supporting appendage can be a foot-supporting appendage incorporating any of the principles, features, limitations, and functions described above with respect to FIGS. 1-11. Indeed, the foot-supporting appendage can include a rearward support configured to support a hind foot portion of a foot of a user, and a frontward support configured to connect to the rearward support and configured to support a forefoot portion of the foot of the user. The methods for modifying a size of a foot-supporting appendage of a wearable robotic exoskeleton can generally include identifying the rearward and frontward supports of the foot-supporting appendage, and sizing the foot-supporting appendage to a size of the foot of the user by modifying, in one or more ways, one or more of the rearward or frontward supports of the foot-supporting appendage to fit a size of the foot of the user of the wearable exoskeleton. As discussed above, modifications of the foot-supporting appendage to accommodate a foot of a current user can comprise interchanging one or more of the frontward or rearward supports of the foot-supporting appendage, such as interchanging a frontward support of one size for another one of a different size. Or, in another example, modifications can comprise unlocking any locking mechanism operable with the foot-supporting appendage and adjusting the relative distance between a frontward support and a rearward support of the foot-supporting appendage that are moveably coupled to one another, and that are moveable relative to one another (e.g., sliding the frontward support forward or backward relative to the rearward support to achieve the correct size to accommodate the user).

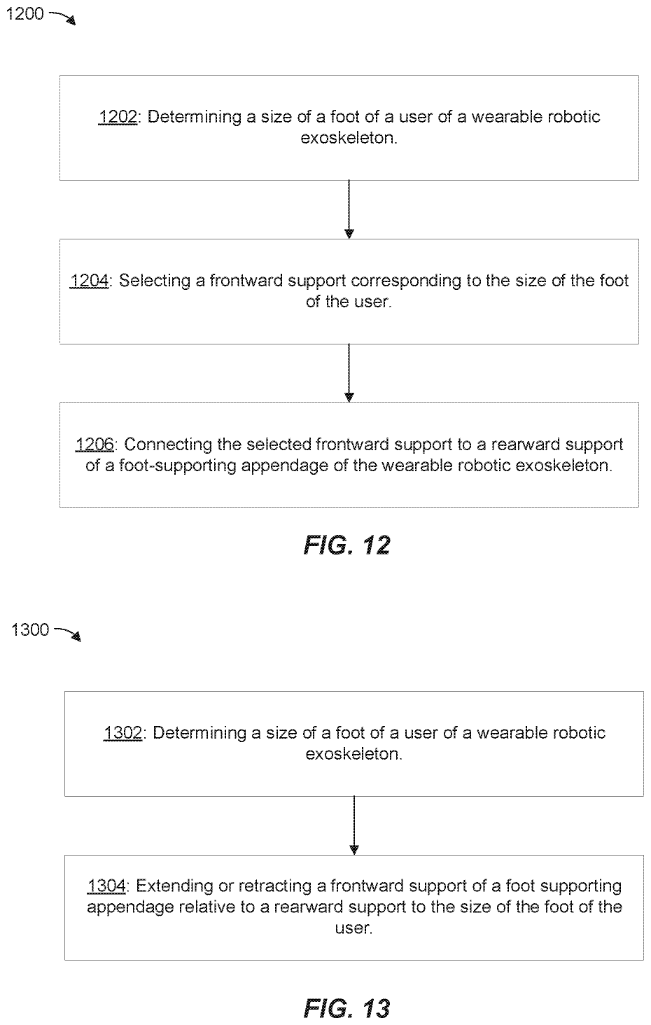

For example, FIG. 12 shows a method 1200 for modifying a size of a foot-supporting appendage of a wearable robotic exoskeleton (e.g., the wearable robotic exoskeleton 100 of FIG. 1) to fit a size of the foot of the user by selecting one of a plurality of interchangeable frontward supports to fit a size of the foot of a current user of the wearable exoskeleton. The method 1200 can include a step 1202 of determining a size of a foot of a user of a wearable robotic exoskeleton. This step can be carried out any number of ways, such as by questioning the user, or by using a measuring device such as a ruler to measure dimensions of the user's foot. This step can also be carried out by simply placing the user's foot in the foot-supporting appendage and visually determining whether the appendage needs to be modified to properly accommodate the user's foot. Method 1200 can further include a step 1204 of selecting a frontward support, from one or more differently sized interchangeable frontward supports that correspond to the size of the foot of the user determined in step 1202. The method 1200 can further include a step 1206 of coupling the selected frontward support to a rearward support of the foot-supporting appendage of the wearable robotic exoskeleton, which can further comprise interchanging the selected frontward support with any existing frontward support already present on the foot-supporting appendage.

In another example, a method for modifying a size of a foot-supporting appendage to accommodate a foot of a user can include steps as follows. FIG. 13 illustrates a method 1300 for modifying a size of a foot-supporting appendage of a wearable robotic exoskeleton (e.g., the wearable robotic exoskeleton 100 of FIG. 1) to fit a size of the foot of the user by adjusting the relative distance between a frontward support and a rearward support of the foot-supporting appendage. Method 1300 can include a step 1302 of determining a size of a foot of a user of a wearable robotic exoskeleton. This step can be carried out any number of ways, such as by questioning the user, or by using a measuring device such as a ruler to measure dimensions of the user's foot. This step can also be carried out by simply placing the user's foot in the foot-supporting appendage and visually determining whether the relative distance between the frontward support and the rearward support of the foot-supporting appendage needs to be adjusted to properly accommodate the user's foot.

Method 1300 can further include a step 1304 of extending or retracting the frontward support of the foot-supporting appendage relative to the rearward support to the size of the foot of the user. This step of extending or retracting of the frontward support of the foot-supporting appendage relative to the rearward support can further comprise actuating an adjustment mechanism configured to facilitate the relative positioning of the rearward support and the frontward support in one or more positions that accommodate a different sized foot of feet of different users. In one example, the adjustment mechanism for extending/retracting the frontward support relative to the rearward support can include one or more rails connected to the rearward support and slideably engaged with the frontward support (e.g., see discussion above regarding FIG. 11), wherein extending or retracting the frontward support relative to the rearward support comprises sliding the frontward support along the one or more rails.

In another example, the adjustment mechanism for extending/retracting the frontward support relative to the rearward support can include one or more threaded rods rotatably engaged with the frontward support and the rearward support (e.g., see discussion above regarding FIG. 9), wherein extending or retracting the frontward support comprises rotating the one or more threaded rods, such as manually via a knob or electronically via a motor.

In still another example, the adjustment mechanism for extending/retracting the frontward support relative to the rearward support can include one or more translational support rods connected to the rearward support and slideably engaged with the frontward support (e.g., see discussion above regarding FIGS. 8A and 8B), wherein extending or retracting the frontward support relative to the rearward support comprises moving (e.g., sliding) the frontward support on the translational rods.

In still another example, the adjustment mechanism for extending/retracting the frontward support relative to the rearward support can include one or more telescoping rods connected to the rearward support and the frontward support (e.g., see discussion above regarding FIGS. 10A and 10B), wherein extending or retracting the frontward support relative to the rearward support comprises extending or retracting the one or more telescoping rods.

In still another example, the adjustment mechanism for extending/retracting the frontward support relative to the rearward support can include one or more rails connected to the frontward support and slideably engaged with the rearward support (e.g., see discussion above regarding FIG. 11), wherein extending or retracting the frontward support relative to the rearward support comprises sliding the rearward support on the one or more rails.

In still another example, the adjustment mechanism for extending/retracting the frontward support relative to the rearward support can include one or more translational rods connected to the frontward support and slideably engaged with the rearward support (e.g., see discussion above regarding FIGS. 8A and 8B), wherein extending or retracting the frontward support relative to the rearward support comprises moving (e.g., sliding) the rearward support on the translational rods.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Although the disclosure may not expressly disclose that some embodiments or features described herein can be combined with other embodiments or features described herein, this disclosure should be read to describe any such combinations that would be practicable by one of ordinary skill in the art. The use of “or” in this disclosure should be understood to mean non-exclusive or, e.g., “and/or,” unless otherwise indicated herein.

Furthermore, the described features, structures, or characteristics can be combined in any suitable manner in one or more examples. In the preceding description, numerous specific details were provided, such as examples of various configurations to provide a thorough understanding of examples of the described technology. It will be recognized, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

1. A foot-supporting appendage of a wearable robotic exoskeleton configured to support a foot of a user wearing the robotic exoskeleton, the foot-supporting appendage comprising:
a rearward support comprising a support surface, and configured to support a hind foot portion of a foot of the user; and
a first frontward support comprising a support surface and a first dimension, the first frontward support being configured to removably couple to the rearward support, and to support a forefoot portion of the foot of the user,
wherein a size of the foot-supporting appendage is adjustable to accommodate a respective foot of a user of a plurality of users having different sizes by removing the first frontward support from the rearward support and attaching to the rearward support a second frontward support having a second dimension different from the first dimension.

2. The foot-supporting appendage of claim 1, wherein a length of the foot-supporting appendage from a rear edge of the rearward support to a front edge of the first frontward support is adjustable.

3. (canceled)

4. (canceled)

5. The foot-supporting appendage of claim 2, wherein the first frontward support is configured to be extendable and retractable relative to the rearward support as part of an adjustment mechanism, the adjustment mechanism comprising at least one of the first frontward and the rearward supports, wherein the adjustment mechanisms facilitates the sizing and adjustability of the foot-supporting appendage.

6. The foot-supporting appendage of claim 5, comprising one or more biasing members coupling the first frontward support to the rearward support that apply a biasing force to the first frontward support to bias the first frontward support toward the rearward support.

7. The foot-supporting appendage of claim 5, further comprising:
one or more rails connected to the rearward support and slideably engaged with the first frontward support;
wherein the first frontward support is extendable and retractable by sliding the first frontward support on the one or more rails.

8. The foot-supporting appendage of claim 5, further comprising:
one or more threaded rods rotatably engaged with the rearward support and the first frontward support;
wherein the first frontward support is extendable and retractable by rotation of the one or more threaded rods.

9. The foot-supporting appendage of claim 5, further comprising:
one or more translational rods connected to the rearward support and slideably engaged with the first frontward support;
wherein the first frontward support is extendable and retractable by actuation of the first frontward support on the one or more translational rods.

10. The foot-supporting appendage of claim 5, further comprising:
one or more telescoping rods connected to the rearward support and the first frontward support;
wherein the first frontward support is extendable and retractable relative to the rearward support by actuation of the one or more telescoping rods.

11. The foot-supporting appendage of claim 5, further comprising:
one or more rails connected to the rearward support and slideably engaged with the first frontward support;
wherein the first frontward support is extendable and retractable relative to the rearward support by sliding the rearward support on the one or more rails.

12. The foot-supporting appendage of claim 5, further comprising:
one or more translational rods connected to the first frontward support and slideably engaged with the rearward support;
wherein the first frontward support is extendable and retractable relative to the rearward support by actuation of the rearward support on the one or more translational rods.

13. The foot-supporting appendage of claim 1, further comprising a load cell configured to provide force signals to a controller of a robotic exoskeleton, the load cell being disposed on a support surface of the foot-supporting appendage where a user's foot interfaces with the foot-supporting appendage.

14. The foot-supporting appendage of claim 13, wherein the load cell is connected to the rearward support of the foot-supporting appendage.

15. The foot-supporting appendage of claim 13, wherein the load cell is a six degree of freedom force-moment load cell.

16. A wearable robotic exoskeleton system comprising:
a foot-supporting appendage configured to support a foot of a user wearing the robotic exoskeleton, the appendage comprising:
a rearward support configured to support a hind foot portion of a foot of the user; and
a first frontward support comprising a first dimension, and configured to removably connect to the rearward support, and configured to support a forefoot portion of the foot of the user;
wherein a size of the foot-supporting appendage is adjustable to accommodate a respective foot of a user of a plurality of users having different sizes of feet by removing the first frontward support having the first dimension from the rearward support and attaching a second frontward support, having a second dimension different from the first dimension, to the rearward support.

17. The wearable robotic exoskeleton system of claim 16, wherein a length of the foot-supporting appendage from a rear edge of the rearward support to a front edge of the first frontward support is adjustable.

18. (canceled)

19. (canceled)

20. The wearable robotic exoskeleton system of claim 17, wherein the first frontward support is configured to be extendable and retractable relative to the rearward support such that the size of foot-supporting appendage is adjustable by extending or retracting the first frontward support relative to the rearward support.

21. The wearable robotic exoskeleton system of claim 20, comprising one or more biasing members coupling the first frontward support to the rearward support that apply a biasing force to the first frontward support to bias the first frontward support toward the rearward support.

22. The wearable robotic exoskeleton system of claim 20, further comprising:
one or more rails connected to the rearward support and slideably engaged with the first frontward support;
wherein the first frontward support is extendable and retractable by sliding the first frontward support on the one or more rails.

23. The wearable robotic exoskeleton system of claim 20, further comprising:
one or more threaded rods rotatably engaged with the rearward support and the first frontward support;
wherein the frontward support is extendable and retractable by rotation of the one or more threaded rods.

24. The wearable robotic exoskeleton system of claim 20, further comprising:
one or more translational rods connected to the rearward support and slideably engaged with the first frontward support;
wherein the first frontward support is extendable and retractable by actuation of the first frontward support on one or more of the translational rods.

25. The wearable robotic exoskeleton system of claim 20, further comprising:
one or more telescoping rods connected to the rearward support and the first frontward support;
wherein the first frontward support is extendable and retractable relative to the rearward support by actuation of the one or more telescoping rods.

26. The wearable robotic exoskeleton system of claim 20, further comprising:
one or more rails connected to the first frontward support and slideably engaged with the rearward support;
wherein the first frontward support is extendable and retractable relative to the rearward support by sliding the rearward support on the one or more rails.

27. The wearable robotic exoskeleton system of claim 20, further comprising:
one or more translational rods connected to the first frontward support and slideably engaged with the rearward support;
wherein the first frontward support is extendable and retractable relative to the rearward support by actuation of the rearward support on the one or more translational rods.

28. The wearable robotic exoskeleton system of claim 16, further comprising a load cell configured to provide force signals to a controller of a robotic exoskeleton, the load cell being disposed on an interface surface of the foot-supporting appendage where a user's foot interfaces with the foot-supporting appendage.

29. The wearable robotic exoskeleton system of claim 28, wherein the load cell is connected to the rearward support of the foot-supporting appendage.

30. The wearable robotic exoskeleton system of claim 28, wherein the load cell is a six degree of freedom force-moment load cell.

31. An adjustable foot-supporting appendage system for a wearable robotic exoskeleton, the system comprising:
a foot-supporting appendage of a wearable robotic exoskeleton configured to support a foot of a user wearing the robotic exoskeleton, the appendage comprising:
a rearward support configured to support a hind foot portion of a foot of the user; and
a plurality of frontward supports configured to interchangeably connect to the rearward support and configured to support a forefoot portion of the foot of the user;
wherein each of the plurality of frontward supports are associated with a foot size of a user;
wherein a size of the foot-supporting appendage is adjustable by removably coupling one of the plurality of frontward supports to the rearward support, the one of the plurality of frontward supports being a size corresponding to a size of foot of the user.

32. A method for modifying a size of a foot-supporting appendage of a wearable robotic exoskeleton, the method comprising:
identifying a rearward support configured to support a hind foot portion of a foot of a user;
identifying a first frontward support comprising a first dimension, and configured to removably couple to the rearward support, and to support a forefoot portion of the foot of the user; and
sizing the foot-supporting appendage to a size of the foot of the user by removing the first frontward support from the rearward support and attaching to the rearward support a second frontward support having a second dimension different from the first dimension.

33. The method of claim 32, wherein the size of the foot-supporting appendage from a rear edge of the rearward support to a front edge of the first frontward support is adjustable.

34. The method of claim 33, wherein the first frontward support is configured to be removably connected to the rearward support, and wherein the sizing of the foot-supporting appendage comprises:
determining the size of the foot of the user;
selecting a second frontward support from a plurality of interchangeable frontward supports that corresponds to a size of the foot of the user; and
coupling the selected second frontward support to the rearward support.

35. The method of claim 32, wherein the first frontward support is configured to be extendable and retractable relative to the rearward support, wherein sizing the foot-supporting appendage comprises:
determining the size of the foot of the user; and
extending or retracting the first frontward support of the foot-supporting appendage relative to the rearward support to the size of the foot of the user.

36. The method of claim 35, wherein extending or retracting the first frontward support of the foot-supporting appendage relative to the rearward support comprises actuating an adjustment mechanism, the adjustment mechanism comprising at least one of the first frontward and the rearward supports.

37. A foot-supporting appendage of a wearable robotic exoskeleton configured to support a foot of a user wearing the robotic exoskeleton, the foot-supporting appendage comprising:
a rearward support comprising a support surface, and configured to support a hind foot portion of a foot of the user; and
a frontward support comprising a support surface, and configured to couple to the rearward support, and to support a forefoot portion of the foot of the user, the frontward support being extendable and retractable relative to the rearward support as part of an adjustment mechanism, that facilitates the sizing and adjustability of the foot-supporting appendage,
wherein a size of the foot-supporting appendage is adjustable to accommodate a respective foot of a user of a plurality of users having different sizes, and
wherein a length of the foot-supporting appendage from a rear edge of the rearward support to a front edge of the frontward support is adjustable.

38. A foot-supporting appendage of a wearable robotic exoskeleton configured to support a foot of a user wearing the robotic exoskeleton, the foot-supporting appendage comprising:
a rearward support comprising a support surface, and configured to support a hind foot portion of a foot of the user; and
a load cell configured to provide force signals to a controller of a robotic exoskeleton, the load cell being disposed on a support surface of the foot-supporting appendage where a user's foot interfaces with the foot-supporting appendage, wherein the load cell is connected to the rearward support of the foot-supporting appendage, and wherein the load cell is a six degree of freedom force-moment load cell,
a frontward support comprising a support surface, and configured to couple to the rearward support, and to support a forefoot portion of the foot of the user,
wherein a size of the foot-supporting appendage is adjustable to accommodate a respective foot of a user of a plurality of users having different sizes.

39. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more rails connected to the rearward support and slideably engaged with the frontward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is configured to be extendable and retractable relative to the rearward support by sliding the frontward support on the one or more rails.

40. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more threaded rods rotatably engaged with the rearward support and the frontward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is extendable and retractable to adjust the size of the foot-supporting appendage by rotation of the one or more threaded rods.

41. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more translational rods connected to the rearward support and slideably engaged with the frontward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is extendable and retractable to adjust the size of the foot-supporting appendage by actuation of the frontward support on one or more of the translational rods.

42. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more telescoping rods connected to the rearward support and the frontward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is extendable and retractable to adjust the size of the foot-supporting appendage by actuation of the one or more telescoping rods.

43. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more rails connected to the frontward support and slideably engaged with the rearward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is extendable and retractable to adjust the size of the foot-supporting appendage by sliding the rearward support on the one or more rails.

44. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
one or more translational rods connected to the frontward support and slideably engaged with the rearward support,
wherein a size of the foot-supporting appendage is adjustable to accommodate a respective foot of a user of a plurality of users having different sizes of feet,
wherein a length of the foot-supporting appendage from a rear edge of the rearward support to a front edge of the frontward support is adjustable,
wherein the frontward support is extendable and retractable to adjust the size of the foot-supporting appendage by actuation of the rearward support on the one or more translational rods.

45. A wearable robotic exoskeleton system comprising:
a foot-supporting appendage configured to support a foot of a user wearing the robotic exoskeleton, the appendage comprising:
a rearward support configured to support a hind foot portion of a foot of the user;
a frontward support configured to connect to the rearward support, and configured to support a forefoot portion of the foot of the user; and
a load cell configured to provide force signals to a controller of a robotic exoskeleton, the load cell being disposed on an interface surface of one of the rearward support or the frontward support where a user's foot interfaces with the foot-supporting appendage, wherein the load cell is a six degree of freedom force-moment load cell,
wherein a size of the foot-supporting appendage is adjustable to accommodate a respective foot of a user of a plurality of users having different sizes of feet.
